(19) United States
(12) Patent Application Publication
(10) Pub. No.: US 2026/0112499 A1
(43) Pub. Date: Apr. 23, 2026

(54) System

(71) Applicant: Softbank Group Corp., Tokyo (JP)

(72) Inventor: Yutaro Miyagawa, Tokyo (JP)

(21) Appl. No.: 19/358,006

(22) Filed: Oct. 14, 2025

(30) Foreign Application Priority Data
Oct. 18, 2024 (JP) 2024-183895

(51) Int. Cl. G16H 50/30

(52) U.S. Cl. CPC G16H 50/30

(57) ABSTRACT

A system includes a processor that is configured to collect biological data of a user and store the data in a database, analyze the stored data and predict the health condition of the user, provide lifestyle guidance to the user based on the prediction result, and issue a warning when an abnormal value is detected.

### CROSS-REFERENCE TO RELATED APPLICATION

This application is based on and claims priority under 35 USC 119 from Japanese Patent Application No. 2024-183895 filed on Oct. 18, 2024, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventionally, effective management of chronic diseases such as diabetes requires patients to frequently monitor various biological parameters, interpret complex data, and make appropriate lifestyle decisions. This process is burdensome and often leads to suboptimal health outcomes due to errors, lack of timely feedback, or failure to recognize abnormal trends. There is a need for a system that can automatically collect relevant health data, provide timely analysis and personalized guidance, and promptly alert users to abnormal conditions in order to support improved disease management and quality of life.

### SUMMARY

The present invention provides a system comprising a processor configured to automatically collect biological data of the user, such as blood glucose levels, meal information, and exercise information, and store the data in a database. The processor further analyzes the stored data to predict the health condition of the user, generates and delivers lifestyle guidance based on the prediction, and issues warnings when abnormal values are detected. Additionally, the system can receive and incorporate user feedback into the analysis, thereby enabling continuous refinement of health management and supporting more accurate and personalized recommendations.

“Processor” means that a hardware component or a combination of hardware and software capable of executing programmed instructions to perform computations and control operations within the system.

“Biological data” means that information relating to the physiological or health characteristics of a user, including but not limited to blood glucose levels, meal information, and exercise information.

“Database” means that a structured collection of data stored electronically, which allows for storage, retrieval, and management of biological data associated with a user.

“Analysis” means that the process of examining, processing, and interpreting biological data to extract meaningful patterns or information, especially to predict the user's health condition.

“Prediction” means that estimating a future or current state of a user's health condition based on analysis of stored biological data.

“Lifestyle guidance” means that information, recommendations, or instructions provided to the user for the purpose of supporting healthy habits, including advice on diet, exercise, or medication adjustments.

“Warning” means that a notification or alert issued to the user when the system detects an abnormal value or condition that may require the user's immediate attention.

“Abnormal value” means that a data point or measurement from the user's biological data that falls outside predefined safe or expected ranges and may indicate a health risk.

“Feedback” means that information provided by the user regarding their response to the system's guidance, actions taken, or subsequent health status, which may be used to improve the system's analysis and recommendations.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

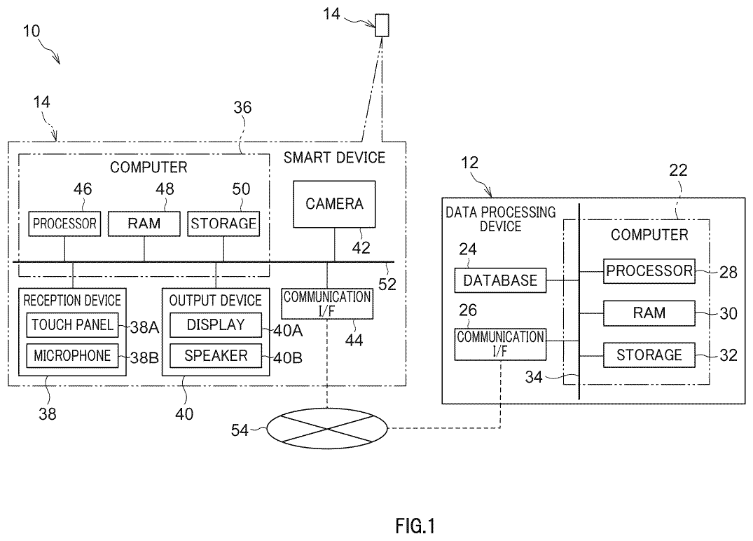

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

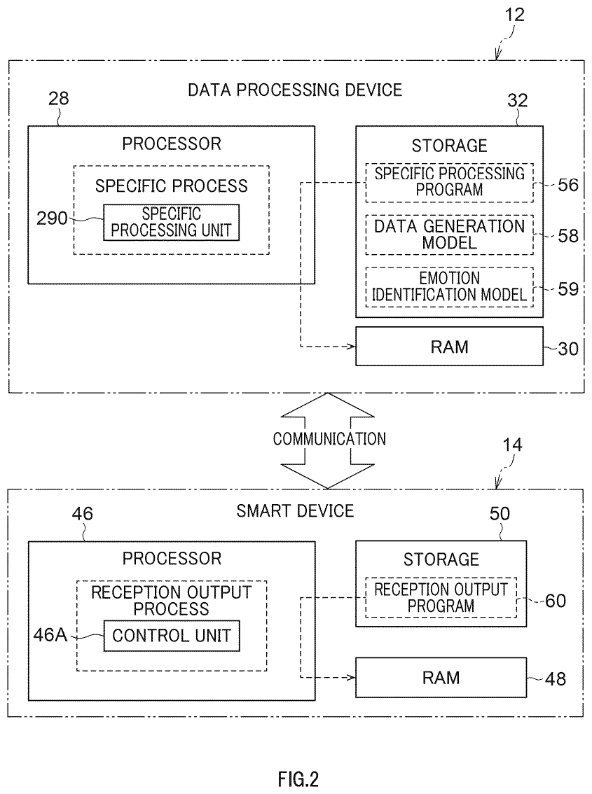

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional health management systems have difficulty in effectively collecting and analyzing biometric information of users and providing appropriate and timely health guidance. In particular, it is challenging to obtain real-time biometric data such as blood glucose levels, analyze the data instantaneously, and immediately provide tailored lifestyle guidance based on prediction of the user's future health state. Furthermore, it is important to reliably detect abnormal biometric values and issue timely warnings to minimize health risks and support optimal health outcomes. There is also a need to continuously improve prediction accuracy and user experience by utilizing feedback from users.

The specific processing by the specific processing unit 290 of the data processing device 12 in Example 1 is realized by the following means.

The present invention provides a server comprising a processor configured to obtain biometric information of a user and transmit the biometric information via a communication apparatus, store the obtained biometric information in a data storage device, generate a prompt sentence for a generative artificial intelligence model based on the stored and past biometric information, execute inference by the generative artificial intelligence model to predict the health state of the user and generate personalized lifestyle guidance, detect abnormal biometric information and issue warning notifications via a user terminal, and use feedback information from the user to improve the generative artificial intelligence model. This enables real-time collection and intelligent analysis of biometric information, immediate notification of health guidance and warnings to the user, and continuous improvement of prediction and guidance accuracy using user feedback.

The term “biometric information” refers to physiological or behavioral data related to a user, such as blood glucose data, nutritional intake information, or physical activity information, which are used to assess the health state of the user.

The term “information processing device” refers to an apparatus or system, such as a server or computer, that receives, stores, and processes biometric information collected from a user.

The term “communication apparatus” refers to a hardware or software component that facilitates the transmission of data between devices, such as data transmission from a user terminal to a server via a wireless or wired network.

The term “data storage device” refers to a memory unit or storage system that retains digital data, including biometric information and user feedback information, for processing and analysis.

The term “generative artificial intelligence model” refers to a computer algorithm or machine learning model, such as a neural network, configured to analyze biometric information, generate predictions regarding the user's future health state, and create personalized lifestyle guidance.

The term “prompt sentence” refers to a text-based input generated based on biometric information, which serves as a query or instruction for the generative artificial intelligence model to produce predictions or recommendations.

The term “prediction data” refers to the output of the generative artificial intelligence model, indicating the expected future health state or risk level of the user, derived from analysis of biometric information.

The term “lifestyle guidance content” refers to advice, recommendations, or notifications generated based on prediction data, intended to guide the user in making healthier daily life decisions.

The term “user terminal” refers to an electronic device, such as a smartphone, tablet, or personal computer, that enables the user to interact with the system, receive notifications, and provide feedback.

The term “feedback information” refers to data input by the user, such as responses, evaluations, or health status updates, which is utilized to retrain and improve the generative artificial intelligence model.

One embodiment of the invention will be described below in detail, based on the above claims.

The system comprises a server equipped with a processor, a data storage device, and a communication apparatus, and a user terminal comprising a measurement device, user interface, and communication capability. The system can be implemented using general-purpose hardware, such as standard servers and smartphones, and may utilize cloud-based server resources. For instance, the server can be realized using a cloud computing platform, such as a virtual machine provided by a cloud service provider, and the data storage device may be a secure database system such as a relational database. The generative artificial intelligence model may be implemented using a neural network framework, for example, by employing software libraries such as TensorFlow or PyTorch.

The user terminal collects biometric information including, but not limited to, blood glucose data, nutritional intake information, and physical activity information. Measurement devices, such as continuous glucose monitoring sensors, wearable activity trackers, or input forms in dedicated smartphone applications, are used. The terminal transmits the acquired biometric information to the server using a secure wireless communication protocol, such as HTTPS.

The server receives the biometric information, stores it in the data storage device, and manages and maintains the information for each user. The server refers to historical biometric information related to the same user, and utilizes both the newly received and stored information to generate a prompt sentence. This prompt sentence is designed to summarize the user's situation and to instruct the generative artificial intelligence model for prediction and personalized guidance.

For example, the prompt sentence may be:
“The user's blood glucose readings over the past three mornings are 110, 125, and 145 mg/dL. Breakfast consisted mostly of bread and jam (about 60g carbohydrates). Suggest personalized advice for efficient carbohydrate management and recommend an optimal time for exercise.” The server inputs the prompt sentence and relevant biometric information into the generative artificial intelligence model. The generative artificial intelligence model, implemented with a machine learning framework, calculates prediction data for the user's health state, such as projected blood glucose levels or health risk indicators, and generates lifestyle guidance content, such as recommendations on diet or exercise.

When an abnormal biometric value is detected (for example, if the blood glucose level surpasses a predefined threshold), the server immediately creates a warning message and notifies the user terminal. The user terminal displays the guidance or warning content via a visual alert, sound, or vibration.

The user receives and reviews the lifestyle guidance, then adjusts behavior accordingly. The user may also provide feedback, such as indicating whether the recommendation was followed or describing resulting health changes, through the terminal's input interface.

The server accumulates the user feedback and biometric outcomes in the data storage device.

Periodically, the server utilizes this feedback information to improve and retrain the generative artificial intelligence model, thereby increasing the system's adaptability and guidance accuracy for future use.

This embodiment enables real-time collection of biometric information, prompt and personalized health prediction and guidance using a generative artificial intelligence model, and continuous improvement of prediction performance through incorporation of user feedback. The entire system is designed to be secure, scalable, and adaptable to a wide range of biometric sensor devices and user interfaces.

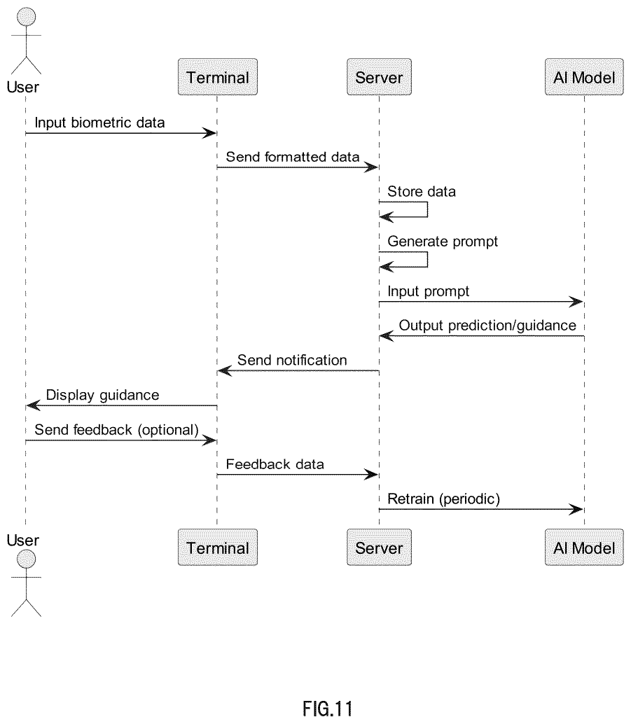

The following describes the processing flow using FIG. 11.
Step 1:

The terminal acquires biometric information from the user by interfacing with measurement devices such as blood glucose sensors, activity trackers, and input interfaces for nutritional data. The input consists of raw sensor readings and user-inputted data such as meal content and exercise logs. The terminal processes these inputs by validating data ranges, associating timestamps, and formatting the information into a standardized digital structure. The output is a formatted and structured data packet containing biometric readings, meal information, and activity details.
Step 2:

The terminal transmits the formatted biometric data packet to the server using a secure communication protocol (such as HTTPS). The input is the structured data packet generated in Step 1. The terminal initiates the data transfer, manages encryption, and handles possible transmission errors. The output is the successful delivery of biometric data to the server and a confirmation message shown on the terminal.
Step 3:

The server receives the biometric data packet and checks the integrity and authenticity of the received information. The input is the encrypted data packet from the terminal. The server verifies the user identity, ensures data is not corrupted, and parses the information for further processing. The output is validated biometric data ready for storage.
Step 4:

The server archives the validated biometric data in a data storage device, such as a database. The input is validated data from Step 3. The server associates data with the user's historical records and commits it to persistent storage, optionally performing backup operations. The output is updated, securely stored user data that is accessible for analysis.
Step 5:

The server retrieves current and historical biometric data for the user and generates a prompt sentence to summarize the user's situation for the generative AI model. The input is a collection of historical and recent biometric records. The server concatenates and formats the data contextually and generates a text-based prompt. The output is a prompt sentence that conveys the user's health trajectory and relevant context.
Step 6:

The server inputs the prompt sentence, along with pertinent biometric data, into the generative AI model. The input is the prompt sentence and associated structured data. The generative AI model processes the input, performs pattern recognition and inference operations, and generates assessment results. The output is a prediction of the user's health state, such as risk levels, and lifestyle guidance content including diet and exercise recommendations.
Step 7:

The server determines whether the AI results indicate abnormal biometric values or health risks. The input is the AI-generated prediction and guidance. The server compares biometrics to predefined thresholds and, if necessary, creates a warning message. The output is either a standard guidance notification or an urgent warning for the user.
Step 8:

The server delivers the lifestyle guidance and any warning notification to the terminal via a push notification service. The input is the AI-generated guidance or warning message. The server formats the notification and sends it to the user's terminal. The output is the receipt and display of guidance or alert on the terminal.
Step 9:

The terminal presents the guidance or warning to the user through visual alerts, sound signals, or vibration cues. The input is the notification from the server. The terminal displays the message, plays audio cues, or vibrates to ensure user attention. The output is the successful presentation of recommendations or warnings to the user.
Step 10:

The user reviews the notification, adjusts daily lifestyle as advised, and may optionally provide feedback via the terminal interface. The input is the system's recommendation and the user's own behavioral response. The user inputs his/her feedback about compliance or results (for example, “I walked after breakfast as suggested”). The output is a new feedback record submitted to the terminal.
Step 11:

The terminal transmits the feedback record to the server using secure data transmission. The input is the feedback from the user. The terminal formats and encrypts the feedback for server submission. The output is the delivery of user feedback to the server's data storage.
Step 12:

The server stores the received feedback in the data storage device and periodically utilizes the accumulated feedback to retrain and improve the generative AI model. The input is the user feedback data. The server incorporates this information into the AI's training datasets, initiating machine learning training jobs as scheduled. The output is an updated generative AI model with improved prediction and recommendation accuracy for future cycles.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

In the field of personal health management, particularly for individuals with chronic conditions such as diabetes, it is difficult to provide timely and personalized support based on a comprehensive and real-time understanding of a user's biometric, behavioral, and emotional status. Conventional systems often lack the ability to integrate emotional data and provide optimized advice, resulting in delayed or inadequate responses to sudden changes in a user's health condition and insufficient adaptation to the individual's psychological state. There is a need for a system that can continuously collect multifaceted user data, perform advanced predictive analysis, and deliver personalized advice and emergency alerts efficiently.

The specific processing by the specific processing unit 290 of the data processing device 12 in Application Example 1 is realized by the following means.

The present invention provides a server comprising a processor configured to collect biometric information, behavioral information, and emotional information of a user; store said information; analyze the stored information using machine learning algorithms and a generative artificial intelligence model; predict a health index and a health condition of the user; generate and transmit an advice sentence based on analysis and prediction results to a terminal device; detect abnormal conditions and issue alerts to medical professionals; and reflect estimated emotional states in health prediction and advice content. This enables timely, individualized health support by integrating comprehensive real-time data analysis and emotional context, thereby improving the safety, adaptability, and quality of life of users with chronic health concerns.

The term “biometric information” refers to data related to measurable physical or physiological characteristics of a user, such as blood component values, blood glucose levels, vital signs, and other health-related indices.

The term “behavioral information” refers to data concerning the actions or habits of a user, including dietary history, exercise history, meal timing, activity patterns, and lifestyle routines.

The term “emotional information” refers to data representing the psychological or emotional states of a user, which are estimated through analysis of voice, facial expressions, written inputs, or other sensors that assess mood or stress levels.

The term “information processing device” refers to any hardware or system, such as a computer, mobile communication device, or embedded system, configured to acquire, process, and transmit user-related information.

The term “memory device” refers to any data storage medium, including local storage, external memory, cloud storage, or database systems, that can save and retain information acquired or generated during system operation.

The term “machine learning algorithm” refers to a computational method or set of techniques capable of learning patterns or making predictions from input data, including but not limited to supervised, unsupervised, or reinforcement learning models.

The term “generative artificial intelligence model” refers to a type of artificial intelligence system designed to generate new outputs—such as advice sentences—based on learned patterns from large datasets, and capable of creating personalized content in response to user conditions and needs.

The term “health index” refers to one or more quantitative or qualitative indicators derived from biometric, behavioral, and emotional information, representing the current or predicted health state of a user.

The term “advice sentence” refers to a textual or audio message automatically generated by the system to provide individualized health-related guidance, recommendations, or instructions to the user based on the prediction and analysis results.

The term “terminal device” refers to a user-side hardware component, such as a smartphone, tablet, smartwatch, or any connected device, capable of receiving advice sentences and transmitting user data or feedback to the server.

The term “medical institution” refers to any organization or facility, such as a hospital, clinic, or healthcare provider, where qualified professionals monitor, evaluate, or intervene based on alerts or abnormal health findings communicated by the system.

The term “feedback information” refers to any response or input provided by the user regarding the advice, actions taken, health outcomes, or perceived effects, which is collected by the system for use in further personalization or model improvement.

One embodiment for implementing the invention is described below. The system is composed of a server, one or more terminal devices, and at least one user.

The terminal is configured as a mobile communication device, such as a smartphone or a smartwatch, equipped with an operating system capable of running application software for health data acquisition. The terminal is connected to various sensors and interfaces, including, but not limited to, a blood glucose sensor, a heart rate monitor, an accelerometer, and a camera or microphone for emotion estimation. Representative examples include a generic blood glucose meter for biometric acquisition, a fitness tracker for activity monitoring, and onboard hardware modules for emotion detection through voice or facial analysis.

The terminal collects biometric information, such as blood component values (e.g., blood glucose measurements), as well as behavioral information, such as details of meal intake and physical activity levels. The user may also manually input information regarding meals consumed, physical activities performed, or subjective impressions through the terminal's user interface. For emotional information, the terminal utilizes the camera, microphone, or text input in conjunction with dedicated emotion recognition software modules. Examples of such software may include a commercial emotion estimation library or an open-source deep learning model trained for voice/emotion classification.

The terminal stores acquired data temporarily in local memory and uses encryption technology, such as Android Keystore or iOS Secure Enclave, to securely transmit the information to the server. The server is constructed on a general-purpose computer system, for example, a cloud-based virtual machine or a physical server device with substantial computational resources and storage capacity.

The server receives incoming data from the terminal and stores it in a database, which may be implemented using general-purpose database software, such as relational database management systems or managed cloud-based storage. The server then preprocesses the received data using data manipulation libraries, for example, Pandas for data cleansing and normalization.

For the analysis and prediction process, the server employs a machine learning algorithm and a generative AI model, which may be developed using frameworks such as TensorFlow or PyTorch. The server applies these models to predict the user's health index and health condition, integrating current and historical biometric, behavioral, and emotional information. Based on the analysis and prediction results, the server generates an advice sentence tailored to the specific user using the generative AI model. The advice sentence is designed to be actionable and easy for the user to understand, and includes recommendations related to health behavior (such as diet, exercise, or stress management), explicitly considering the detected emotional state.

The following example demonstrates a prompt sentence generated by the server:
“Based on your morning glucose of 180 mg/dL and detected stress, we recommend a lunch with low glycemic index foods and a brief relaxation exercise before your meal.”

The server sends the advice sentence to the user's terminal as a notification. The terminal then displays this message to the user through the graphical user interface. The user can respond to or acknowledge the advice via the terminal by entering feedback, such as reporting adherence to the recommendation or entering a subjective feeling.

The terminal acquires the feedback, which may include textual responses, mood selection, or behavioral logs, and transmits this data back to the server. The server stores the received feedback in the database and utilizes it as supplemental learning material when refining the machine learning algorithm and generative AI model, thereby increasing personalization and prediction accuracy over time.

In cases where an abnormal condition is detected, such as a blood glucose measurement exceeding a predefined threshold or a combination of physiological and emotional indicators suggesting a potential health emergency, the server automatically composes and transmits an alert message. This message is sent to a remote specialist or a medical institution, enabling timely professional intervention.

Through the described system, user health management is supported in an individualized, continuous, and adaptive manner. All hardware and software elements referenced above are examples, and substitutions or modifications may be implemented according to technological advances or practical requirements.

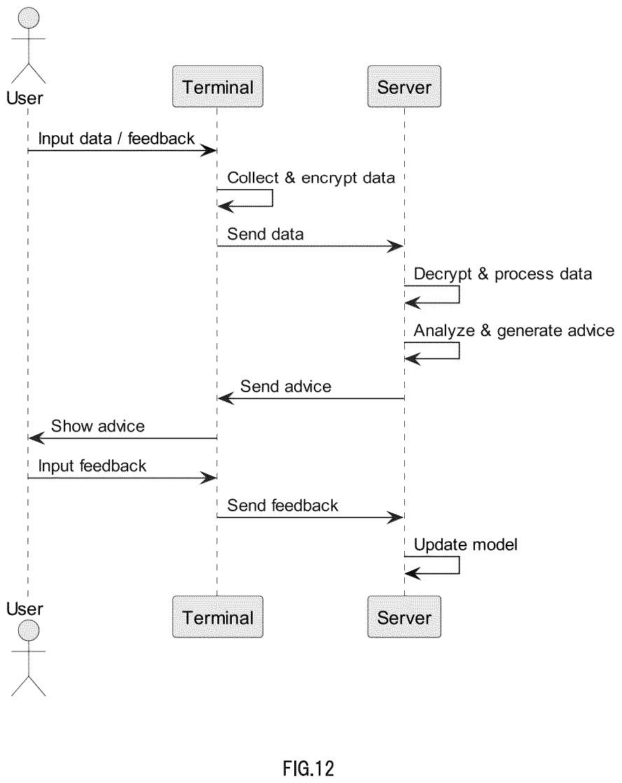

The following describes the processing flow using FIG. 12.
Step 1:

Terminal collects biometric information such as blood glucose readings through a sensor interface, behavioral information such as meal records and physical activity through connected applications, and emotional information by analyzing the user's facial features or voice using emotion recognition software. The input to this step is real-time sensor data, user manual entries, and multimedia input (images or audio). The terminal processes these inputs by aggregating and pre-formatting the data into structured records. The output is an encrypted and organized data record stored in the terminal's local memory.
Step 2:

Terminal encrypts the structured data record by applying a cryptographic method using a secure processing module, such as a secure enclave or keystore. The input is the raw collected data from step 1. The terminal performs data encryption and data packaging processes. The output is an encrypted data package ready for transmission to the server.
Step 3:

Terminal establishes a secure communication channel, such as HTTPS, and transmits the encrypted data package to the server. The input is the encrypted data package. The output is the successful delivery and receipt confirmation of the data at the server side.
Step 4:

Server receives, decrypts, and authenticates the incoming data package using its own cryptographic keys and authentication protocols. The input is the encrypted data package from the terminal. The server processes the data by decrypting and validating the user identity and data integrity. The output is a validated set of structured biometric, behavioral, and emotional data, stored in the server's database.
Step 5:

Server performs preprocessing on the validated data by cleaning, normalizing, and integrating it with historical user information. The input is the structured user data from step 4 and any previously stored user data. The server processes this data by eliminating duplicates, organizing records by temporal sequence, and converting values into a model-compatible format. The output is a preprocessed dataset suitable for analysis.
Step 6:

Server applies a machine learning algorithm and a generative AI model to the preprocessed dataset to analyze the user's current condition, predict a health index, and detect abnormal patterns. The input is the preprocessed dataset from step 5. The server performs data inference, pattern recognition, and risk assessment operations. The output is a set of analytical results, including a health condition prediction, risk indications, and differentiated guidance parameters.
Step 7:

Server generates a prompt sentence using the generative AI model based on analytical results and customizes the advice for the individual user. The input is the set of analytical and predictive outputs from step 6. The server processes these by generating natural language advice, such as “Based on your morning glucose of 180 mg/dL and detected stress, we recommend a lunch with low glycemic index foods and a brief relaxation exercise before your meal.” The output is the generated advice message and associated instructions.
Step 8:

Server transmits the generated advice message to the terminal via a push notification service. The input is the generated advice message from step 7. The output is the delivery of the advice message to the terminal.
Step 9:

Terminal receives the advice message and displays it to the user through a user interface, such as a popup notification or dashboard screen. The input is the advice message from the server. The terminal processes the display and options for user response. The output is the visual or audio presentation of the advice to the user.
Step 10:

User reviews the advice, takes action as recommended (such as following meal, exercise, or relaxation guidance), and may provide feedback regarding adherence or effect. The input is the advice presentation and the user's real-world experience or perception. The user performs an evaluation and manual entry of feedback, if desired. The output is the feedback data entered into the terminal.
Step 11:

Terminal structures, encrypts, and transmits the feedback data to the server for further use. The input is the user feedback data. The terminal processes this by data validation, formatting, and encryption. The output is a feedback data package sent to the server.
Step 12:

Server receives, decrypts, and stores the feedback data. Server uses this feedback to retrain the generative AI model and update personalization parameters for the specific user. The input is the feedback data package from the terminal. The server processes this by integrating new feedback with historical data and performing iterative model training or parameter adjustment. The output is an updated system state and improved predictive and advisory capacity for subsequent operations.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional health management systems for users with chronic conditions, such as diabetes, primarily focus on the collection and analysis of biological data. However, these systems do not sufficiently incorporate the user's emotional state, which can have a significant impact on health outcomes. As a result, they cannot provide optimally personalized lifestyle guidance or accurately predict the user's health condition based on both physiological and psychological factors. Additionally, such systems are often unable to deliver dynamic, situation-adapted recommendations and do not utilize advanced artificial intelligence or user feedback to improve over time. There is a need for a comprehensive system that can integrate biological and emotional data, analyze it using advanced artificial intelligence, and provide personalized guidance that adapts based on user state and feedback.

The specific processing by the specific processing unit 290 of the data processing device 12 in Example 2 is realized by the following means.

The present invention provides a server comprising a processor configured to acquire biological information and emotional state information of a user and store the information in a storage unit, perform integrated analysis on the stored information using a generative artificial intelligence model with historical data to predict health conditions, generate and transmit personalized lifestyle management guidance based on the prediction and the user's emotional state, detect abnormalities in biological or emotional data and provide notifications, and automatically generate input sentences for the generative artificial intelligence model according to situational context, wherein the processor is further configured to collect user feedback and utilize it to refine subsequent analyses and recommendations. This enables comprehensive, personalized health management that dynamically adapts to both physical and emotional states of the user, and continuously evolves its guidance based on user-specific patterns and responses.

The term “biological information” refers to data related to the physiological condition of a user, including but not limited to measurement information, intake information, and activity information.

The term “measurement information” refers to quantitative data obtained from monitoring devices, such as blood glucose levels, blood pressure, heart rate, or other physiological metrics.

The term “intake information” refers to data regarding the amount and type of food, fluids, or medications consumed by the user.

The term “activity information” refers to records of the user's physical movements or exercise, including types, durations, and intensities of physical activity.

The term “emotional state information” refers to data that represents the psychological or emotional condition of a user, determined from sources such as facial expressions, voice analysis, and self-reported feelings.

The term “storage unit” refers to a hardware or software component capable of storing and retrieving digital information, such as a memory device, a storage medium, or a database.

The term “generative artificial intelligence model” refers to a computational model that utilizes machine learning algorithms to process input data and generate predictive analysis and recommendations, based on learned patterns from historical datasets.

The term “historical information” refers to previously collected biological and emotional state data associated with a user, used to inform future predictions and recommendations.

The term “personalized lifestyle management guidance” refers to advice, instructions, or recommendations uniquely tailored to a user's physiological and emotional state, in order to improve health outcomes or address specific needs.

The term “terminal” refers to a user-operated electronic device that communicates with the server, such as a smartphone, tablet, or other portable computing apparatus capable of receiving notifications and displaying information.

The term “warning notification” refers to an alert or message generated when abnormal biological information or emotional state information is detected, intended to prompt user intervention or awareness.

The term “input sentence for the generative artificial intelligence model” refers to a textual or data-based prompt specifically formulated to invoke analysis or recommendations from the generative artificial intelligence model, tailored according to the user's current context and data.

The term “response information” refers to data or feedback provided by the user regarding the utility, effect, or relevance of the advice or notifications received.

An embodiment for implementing the invention is now described below.

The system comprises a server, a terminal used by a user, and various sensors and interfaces for acquiring relevant data.

The terminal is a computing device such as a smartphone or tablet equipped with a display, microphone, front camera, and communication interface (for example, wireless LAN or cellular network module). The terminal is also configured to connect with measurement devices such as a blood glucose sensor or other health monitoring sensors via technologies such as Bluetooth or near-field communication.

The terminal is further equipped with an application program capable of guiding the user to enter information, such as meal data and physical activity, through a user interface. Example application platforms include mobile operating systems such as Android or iOS. The terminal can process and temporarily store collected information using its local memory units.

The terminal is also configured with software libraries and frameworks to process emotional state information. Example software components may include a face detection library (e.g., an open-source face analysis SDK) for image-based emotion recognition and a speech-to-text or voice tone analysis engine for voice-based emotion estimation.

The terminal is configured to securely transmit the acquired biological and emotional state information to the server using an encrypted communication protocol such as HTTPS with TLS.

The server includes a hardware processor, storage unit, and a database management system.

The server may be constituted on a cloud platform (for example, a virtual computing instance or dedicated hardware server in a data center) and may employ a relational or non-relational database for persistent storage of the collected data.

The server is programmed with an integrated data analysis module that includes a generative artificial intelligence model implemented with machine learning frameworks such as PyTorch or TensorFlow. The server performs integrated analysis of the user's biological and emotional state data, as well as relevant historical data stored in the database, by constructing a prompt sentence to query the generative artificial intelligence model.

A typical prompt sentence could be:
“The user has a blood glucose of 142 mg/dL, recently ate oatmeal and milk (350 kcal), walked 20 minutes, and is detected as slightly stressed (stress level 0.7). What morning relaxation advice and dietary adjustments should be suggested?”

The server then generates personalized guidance for the user based on the output of the artificial intelligence model. The guidance is transmitted back to the terminal and may include medical, dietary, or exercise suggestions, as well as recommendations tailored to the user's current emotional state.

The terminal receives the guidance and notifies the user through the application interface or by converting the guidance text to speech using a text-to-speech engine. The user may then act upon the guidance. The user is also prompted to provide feedback about the utility or effect of the advice.

The terminal collects the feedback and transmits it securely to the server. The server uses the feedback to improve future recommendations by updating analysis parameters or retraining the generative artificial intelligence model if required.

Specific examples of hardware and software that can be used in this system include measurement devices such as generic blood glucose meters; terminal devices such as general-purpose smartphones or tablets; communications protocols such as HTTPS; cloud-based databases and servers; software libraries for face and voice analysis; text-to-speech engines; and machine learning frameworks for the generative artificial intelligence model.

The above embodiment enables comprehensive, individualized health management by integrating biological and emotional data, protecting data during transmission, utilizing advanced artificial intelligence for analysis, and enabling a feedback loop for continuous improvement in guidance. This system allows users to receive lifestyle recommendations that are responsive to both their physiological and psychological conditions.

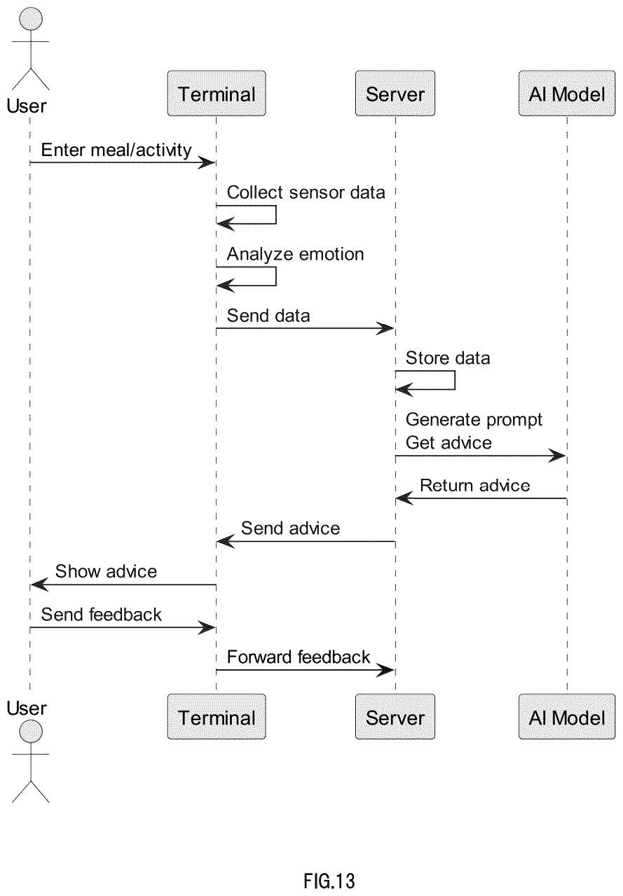

The following describes the processing flow using FIG. 13.
Step 1:

The terminal activates and connects with measurement devices such as a blood glucose sensor via Bluetooth or NFC to acquire biological data. The terminal also prompts the user to manually enter intake information such as meal contents and physical activity through a graphical user interface in the application. The input for this step is sensor output data (e.g., blood glucose level), and user-entered data (e.g., meal and exercise details). The terminal then stores this collected information in local memory. As data processing, the terminal validates the completeness and plausibility of all entries and timestamps each record. The output is a structured dataset containing biological information with associated timestamps.
Step 2:

The terminal requests permission from the user to access the microphone and front camera, then records a short audio and video segment. The input is live audio and image data captured from the user. The terminal uses face detection and voice analysis software to extract emotional state features from the collected media. Data processing includes converting speech to text, analyzing voice tone, and detecting facial expressions. The output is encoded emotional state information, such as a quantified stress or happiness level.
Step 3:

The terminal aggregates the biological and emotional state information into a structured message. The terminal then initiates a secure communication session, encrypts the message, and transmits it to the server via HTTPS. The input is the data set containing biological and emotional information; the data processing includes serializing to a transmission format and performing encryption. The output is an encrypted data payload sent to the server.
Step 4:

The server receives the encrypted data and performs authentication and integrity checks. The input is the encrypted payload from the terminal. The server then decrypts the payload, parses the biological and emotional data, and stores them in a database with a unique user identifier and timestamp. The data processing involves decryption, deserialization, and structured storage in a persistent database. The output is updated user data stored in the database.
Step 5:

The server collects historical data for the user from the database and combines it with newly received data. The server constructs a prompt sentence for the generative AI model. For example, the prompt might be “The user has a blood glucose of 142 mg/dL, recently ate oatmeal and milk (350 kcal), walked 20 minutes, and is detected as slightly stressed (stress level 0.7). What morning relaxation advice and dietary adjustments should be suggested?” The input is the compiled set of historical and current biological and emotional data; the data processing involves prompt generation and formatting. The output is a text prompt suitable for AI processing.
Step 6:

The server supplies the prompt sentence to the generative AI model, which analyzes the input and returns a recommendation. The input is the prompt sentence; data processing by the generative AI model includes evaluating biological and emotional information in context and generating optimized lifestyle advice. The output is an AI-generated recommendation or plan for lifestyle management tailored to the user's current situation.
Step 7:

The server reviews the AI output, merges it with rule-based medical guidelines if necessary, and formats it for transmission to the terminal. The input is the AI-generated lifestyle guidance; data processing involves any required adjustment or filtering for user safety or regulatory compliance. The output is a finalized guidance message.
Step 8:

The server transmits the finalized guidance message to the terminal. The terminal receives the message, presents it to the user through a notification or visual/audio output, and logs the communication. The input is the guidance message; data processing on the terminal includes decoding and appropriate presentation using a display or text-to-speech engine. The output is the communicated guidance shown or read to the user.
Step 9:

The terminal presents an optional feedback prompt to the user regarding the usefulness of the guidance. The user may submit feedback through a selection or text entry. The input for this step is the user's feedback entry; the terminal processes and transmits the feedback data back to the server. The output is the feedback data stored for future analysis.
Step 10:

The server receives feedback data, associates it with the relevant session or guidance, and utilizes it to improve future recommendations. The input is the user feedback data; the server may process it for statistical trend analysis, model retraining, or prompt refinement. The output is continually refined AI model performance and future advice personalization.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

There is a need to provide a system capable of collecting and integrating a user's biometric and emotional information, analyzing these data to predict the user's health status and behavioral tendencies, and generating personalized lifestyle improvement plans or product recommendations based on both biometric and emotional state. Existing systems do not sufficiently consider emotional information in conjunction with biometric data, and cannot flexibly generate prompt sentences for generative models or adaptively recommend health-related products in real time. Moreover, systems that improve through user feedback while ensuring secure transmission and processing of sensitive data are lacking.

The specific processing by the specific processing unit 290 of the data processing device 12 in Application Example 2 is realized by the following means.

The present invention provides a server comprising a processor configured to collect and store biometric information and emotional information of a user, analyze the stored information to predict the user's health status and behavioral tendencies, generate personalized behavioral improvement plans or health-related product recommendations using a generative model based on both the analysis and emotional state, issue warnings in response to abnormal values, securely transmit and receive information, and automatically generate and input prompt sentences to the generative model based on collected information. This enables real-time, adaptive, and personalized health management support that flexibly integrates biometric and emotional data, and continuously improves recommendation quality through user feedback in a secure environment.

The term “biometric information” refers to physiological or biological data of a user, including but not limited to blood component measurement values, dietary intake information, and physical activity information, which are used to evaluate the health status of the user.

The term “emotional information” refers to data indicative of a user's psychological or emotional state, derived from sources such as voice, facial expressions, or other behavioral cues.

The term “storage device” refers to a general-purpose or dedicated data storage unit, such as memory or a database, in which collected information is securely recorded and maintained for later analysis.

The term “analysis device” refers to a processor or computational component configured to analyze biometrical and emotional data for the purpose of predicting user health status and behavioral tendencies.

The term “generative model” refers to an artificial intelligence model, including but not limited to neural network-based machine learning models, which generates output data, such as behavioral improvement plans or product recommendations, based on inputted user data.

The term “behavioral improvement plan” refers to a set of actions, advice, or strategies generated for the user with the purpose of improving lifestyle habits or health outcomes, tailored according to the analysis of biometric and emotional information.

The term “health-related product recommendation information” refers to data generated for the purpose of suggesting products, goods, or services that are beneficial to a user's health condition, which are selected based on a user's current state derived from collected data.

The term “communication terminal” refers to an electronic device, such as a smartphone, tablet, or computer, used for sending or receiving data between the user and the server.

The term “secure communication means” refers to any communication protocol or technology that reliably ensures the confidentiality and integrity of data transmitted between system components, such as encrypted communication channels.

The term “prompt sentence” refers to a textual instruction or query automatically generated and input to the generative model to obtain required output, based on the user's current data and circumstances.

The term “response information” refers to any data indicating the user's reaction to provided recommendations or outputs, including feedback, preferences, or selection results.

The term “learning of the generative model” refers to the process of updating or adapting the generative model based on collected response information, improving its prediction or recommendation accuracy over time.

One embodiment of the invention will now be described in detail, relating to a system for personalized health management and recommendation based on integrated biometric and emotional information.

The system comprises a server, at least one user terminal, and a communication network connecting them. The server includes a processor, storage device (for example, a database), and interfaces for secure communication. The user terminal may be a general-purpose mobile device, such as a smartphone or tablet, equipped with biometric sensors (for example, a blood component measurement device or wearable sensor), a camera, and a microphone.

The user terminal acquires biometric information such as blood glucose values, dietary intake, and physical activity through integrated or connected sensors (e.g., a continuous glucose monitor and accelerometer). The user terminal also records the user's voice and facial expressions using built-in input devices, such as a camera and a microphone. The terminal executes emotion recognition software, for instance, a facial analysis library or a tone analysis module, to estimate the user's emotional state from the acquired data.

The terminal preprocesses and formats the biometric and emotional information, then securely transmits the information to the server over an encrypted communication channel, such as a network using TLS/SSL.

The server receives and stores the data in a storage device, such as a database constructed using a general-purpose database management system. The server analyzes both current and historical biometric and emotional information, employing an analysis device such as a general processor running artificial intelligence software. In a preferred implementation, the server uses a neural network-based generative AI model (such as one created with a machine learning library) to analyze the data and generate outputs.

For generating health-related recommendations, the server composes a prompt sentence according to the user's current biometric and emotional status. The server automatically generates and inputs this prompt sentence into the generative AI model. For example, the prompt input to the model may be:

Based on the user's current blood glucose level and emotional state, please suggest product candidates that can relieve stress.

The generative AI model outputs recommendations or behavioral improvement plans, which the server packages into a personalized message. If the analysis detects abnormal values, the server can generate and transmit a warning to the user terminal.

The user terminal receives the output from the server and presents the result to the user via a user interface. This may include health guidance, a behavioral improvement plan, or a health product recommendation. For example, when the model detects elevated stress and blood glucose, the terminal may display a recommendation such as:

You seem to be experiencing stress and your blood glucose is elevated. We recommend herbal tea and a short walk to support your health. Click here to purchase the suggested product.

The user is able to provide feedback or select from among recommended actions or products using the terminal's interface. The terminal collects the feedback and transmits it to the server, where it is incorporated by the processor for future analysis and optimization. The generative AI model continuously improves the quality and personalization of its recommendations by updating learning parameters based on user feedback.

Throughout this operation, all personal and sensitive information is communicated and processed with secure encryption to maintain confidentiality and data integrity. The system components may employ general hardware and commonly available software for implementation, such as generic smart devices as terminals, general-purpose servers, emotion analysis software, machine learning libraries, and any suitable secure communication protocol.

This embodiment enables real-time, adaptive health management and product recommendation tailored to the user's biometric and emotional status, reliably supporting improved health outcomes and quality of life.

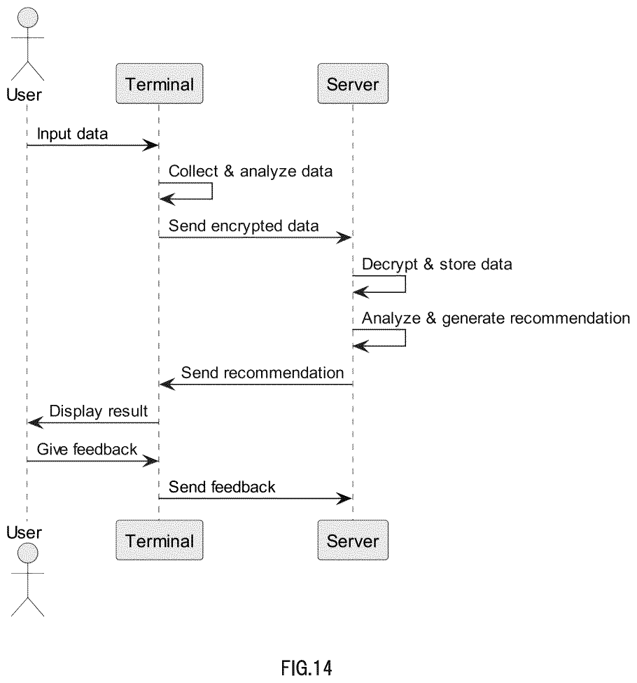

The following describes the processing flow using FIG. 14.
Step 1:

The terminal collects biometric information from the user using integrated sensors, such as a blood glucose monitor, and acquires emotional information from the user by recording voice data with a microphone and capturing facial images with a camera. The terminal applies emotion detection software to analyze the voice and facial data to estimate the user's emotional state. Input: raw sensor data, voice, and image data. The terminal processes the raw inputs to output formatted biometric and emotional data in a standardized structure.
Step 2:

The terminal temporarily stores the formatted biometric and emotional information in local memory. The terminal verifies the completeness and consistency of the collected data, for example, by checking for proper timestamps or missing entries. Input: formatted biometric and emotional data. The terminal outputs a verified data record ready for secure transmission.
Step 3:

The terminal encrypts the verified data record using standard encryption techniques, such as AES-256. The terminal then transmits the encrypted data to the server over a secure network connection utilizing protocols such as HTTPS (TLS/SSL). Input: verified data record. The terminal outputs an encrypted data packet sent to the server.
Step 4:

The server receives the encrypted data packet, decrypts it, and parses the received content to extract biometric and emotional information. The server checks data integrity using checksum validation. Input: encrypted data packet. The server outputs a validated and parsed data record stored in the database.
Step 5:

The server aggregates the newly stored data with historical user data retrieved from the database. The server analyzes the combined dataset to identify patterns, risk factors, or abnormal values using a machine learning algorithm. Input: parsed data record and related historical data. The server outputs preliminary analysis results, such as risk assessments or identification of anomalies.
Step 6:

The server automatically generates a prompt sentence based on the user's current biometric and emotional states. The server inputs this prompt into a generative AI model to generate recommendations or behavioral plans. For example, the prompt might be: Based on the user's blood glucose and emotional state, please recommend suitable products to relieve stress. Input: analysis results, including current biometric and emotional information. The server outputs a personalized recommendation or plan.
Step 7:

The server sends the generated recommendation or plan to the terminal as a formatted message. Input: recommendation or plan generated by the AI model. The server outputs a message delivered to the user's terminal.
Step 8:

The terminal presents the recommendation or plan to the user through its interface, providing options for action, such as direct product purchase or feedback submission. Input: received recommendation or plan message. The terminal outputs a displayed message with actionable buttons.
Step 9:

The user reviews the recommendation, selects an action (such as purchasing a recommended product), or provides feedback on the suggestion. Input: displayed message and user interaction. The user's action results in feedback or a transaction request sent from the terminal to the server.
Step 10:

The terminal collects the user's feedback or transaction result and transmits this information to the server. Input: user feedback data or transaction result. The terminal outputs updated information for server-side analysis or generative model learning.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

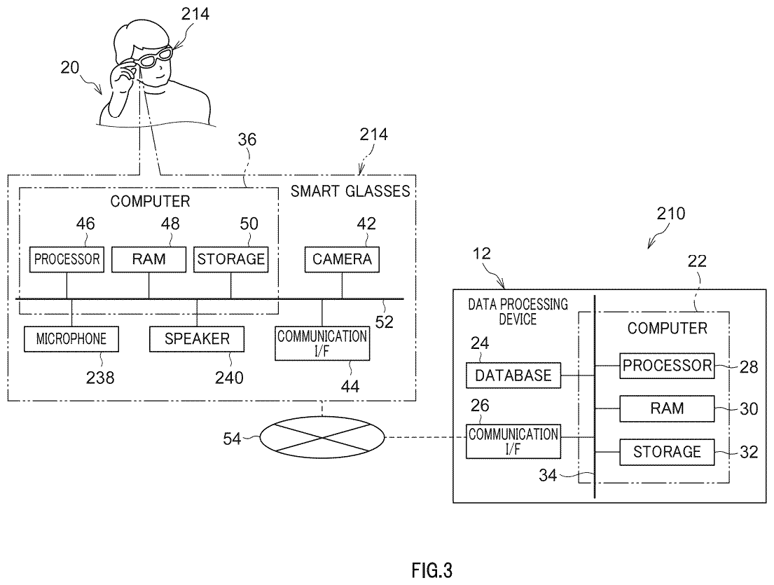

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

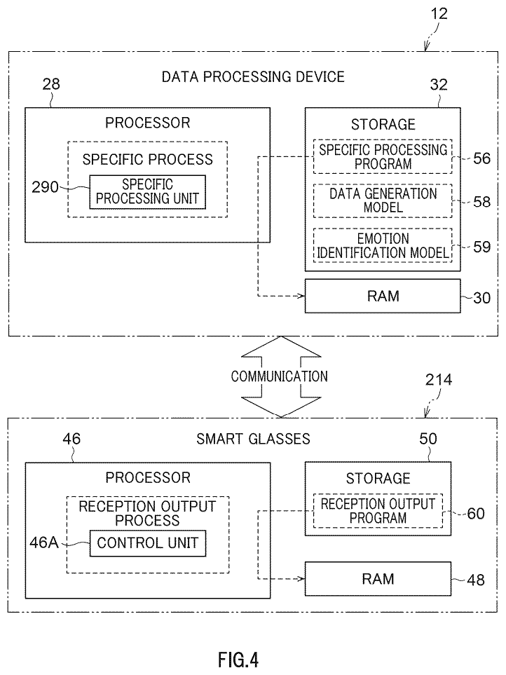

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

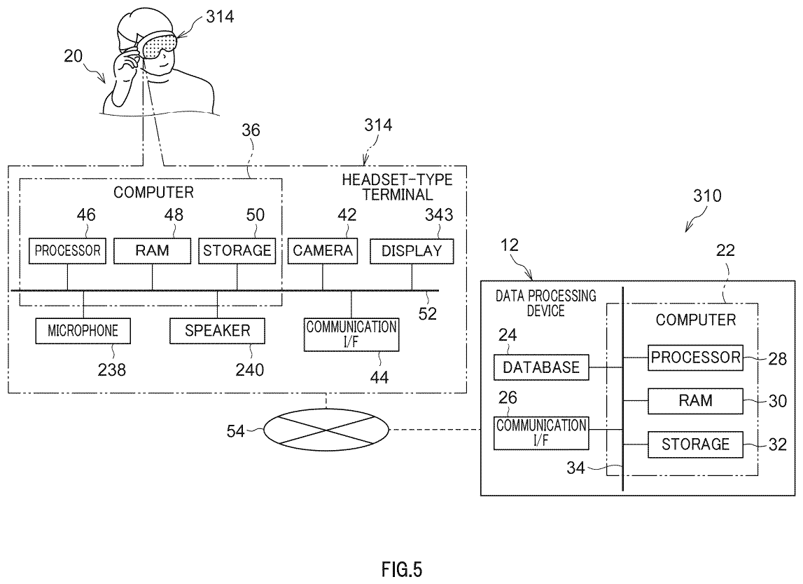

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

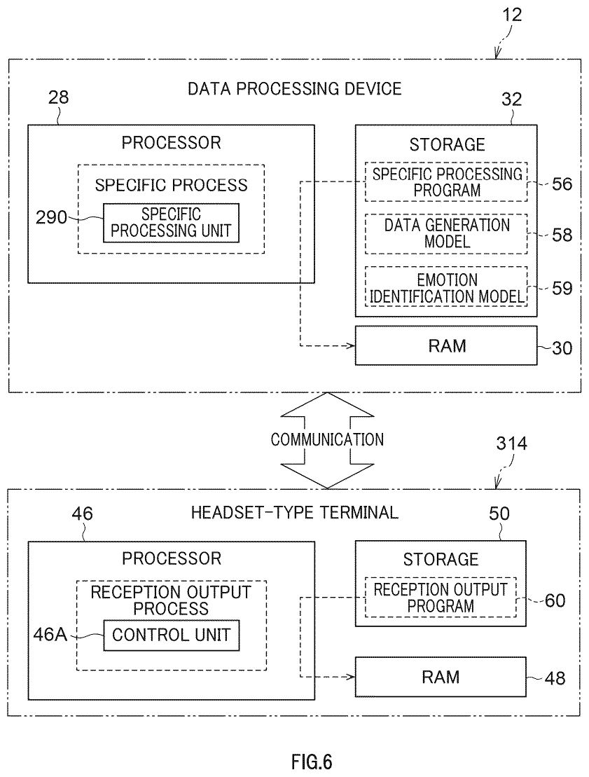

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

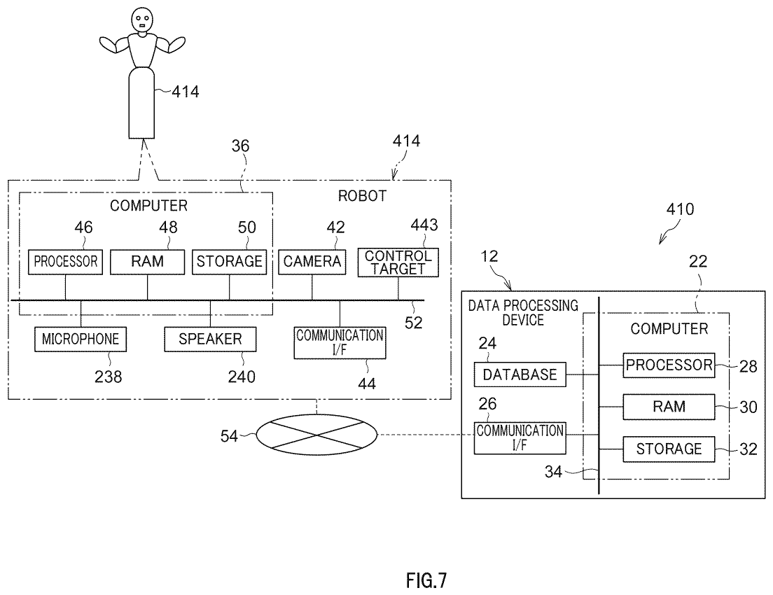

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

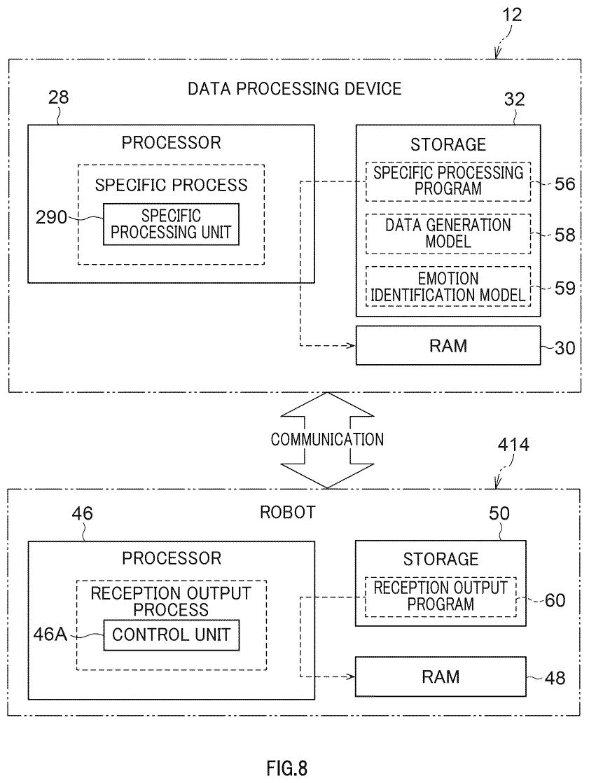

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naive Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

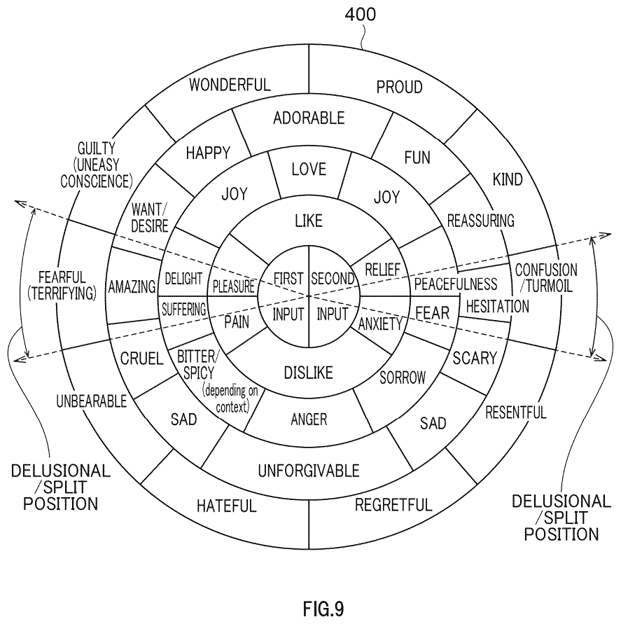

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https: //ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

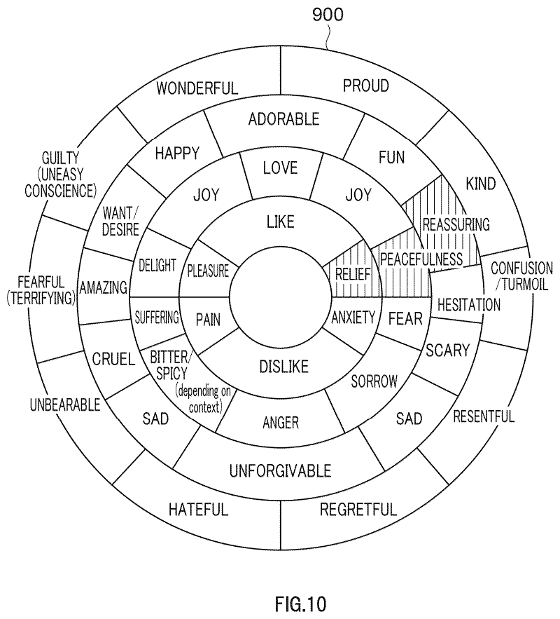

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
obtain biometric information of a user and transmit the biometric information to an information processing device via a communication apparatus,
store the obtained biometric information in a data storage device,
generate a prompt sentence for a generative artificial intelligence model using the stored biometric information and previously obtained biometric information, execute inference by the generative artificial intelligence model, generate prediction data about the user's health state and generate lifestyle guidance content based on the prediction,
detect abnormal biometric information and notify warning information to a user terminal,
and enable notification of the lifestyle guidance content via the user terminal.

### Supplementary 2

The system according to supplementary 1,
wherein the processor is configured to
store feedback information obtained from the user in the data storage device and use the feedback information for training the generative artificial intelligence model.

### Supplementary 3

The system according to supplementary 1,
wherein the biometric information comprises blood glucose data, nutritional intake information, and physical activity information.

### Application Example 1

### Supplementary 1

A system comprising a processor,
wherein the processor is configured to:
collect biometric information, behavioral information, and emotional information of a user by utilizing an information processing device and store the information in a memory device;
analyze the stored information using a machine learning algorithm and a generative artificial intelligence model to predict a health index and a health condition of the user;
generate, based on the analysis and prediction results, an advice sentence optimized for each user by using the generative artificial intelligence model and notify the advice sentence via a terminal device;
detect an abnormal condition exceeding a predetermined threshold from the analyzed information and, in such case, transmit information immediately to a remote specialist or medical institution to issue an alert;
and reflect the estimated emotional state of the user in the prediction of the health condition and the content of the advice.

### Supplementary 2

The system according to supplementary 1,
wherein the processor is configured to acquire response information or feedback information from the user via the terminal device, store the acquired information in the memory device,
and reuse the information in the analysis by the machine learning algorithm and the generative artificial intelligence model in order to improve personalization and accuracy of the advice.

### Supplementary 3

The system according to supplementary 1,
wherein the processor is configured to include, as the biometric information, blood component values, dietary history, exercise history, and emotional estimation results.

### Example 2

### Supplementary 1

A system comprising a processor,
wherein the processor is configured to
acquire biological information and emotional state information of a user and store the information in a storage unit,
perform integrated analysis of the stored biological information and emotional state information using a generative artificial intelligence model with historical information to predict a health condition of the user,
generate personalized lifestyle management guidance based on the predicted health condition and the emotional state of the user and transmit the guidance to a terminal,
detect abnormal biological information or emotional state and provide a warning notification, and
automatically generate an input sentence for the generative artificial intelligence model according to the situation.

### Supplementary 2

The system according to supplementary 1,
wherein the processor is configured to
collect response information from the user and reflect the response information in the analysis by the generative artificial intelligence model.

### Supplementary 3

The system according to supplementary 1,
wherein the processor is configured to
include measurement information, intake information, and activity information as the biological information.

### Application Example 2

### Supplementary 1

A system comprising a processor,
wherein the processor is configured to
collect biometric information and emotional information of a user, and store the information in a storage device;
analyze the stored biometric information and emotional information using an analysis device to predict a health status and behavioral tendencies of the user;
generate, based on the prediction result and the emotional information, a behavioral improvement plan or health-related product recommendation information using a generative model, and provide the information to the user via a communication terminal;
issue a warning when an abnormal value exists in the analysis result or biometric information; transmit and receive information using a secure communication means;
automatically generate a prompt sentence corresponding to the input information and input the prompt sentence into the generative model.

### Supplementary 2

The system according to supplementary 1,
wherein the processor is configured to
collect response information or selection results from the user and utilize the response information or selection results for the analysis or learning of the generative model.

### Supplementary 3

The system according to supplementary 1,
wherein the processor is configured to
include blood component measurement values, dietary intake information, and physical activity information as the biometric information.

What is claimed is:

1. A system comprising a processor,
the processor being configured to:
collect biological data of a user and store the data in a database;
analyze the stored data and predict the health condition of the user,
provide lifestyle guidance to the user based on the prediction result; and
issue a warning when an abnormal value is detected.

2. The system according to claim 1, wherein the processor is further configured to collect feedback from the user and utilize the feedback in the analysis.

3. The system according to claim 1, wherein the biological data includes blood glucose levels, meal information, and exercise information.
